(19) United States
(12) Patent Application Publication
(10) Pub. No.: US 2026/0248934 A1
(43) Pub. Date: Aug. 27, 2026

(54) CANCER TREATMENT WITH A PD-1 OR PD-L1 INHIBITOR AND AN ANTIBODY-DRUG CONJUGATES TARGETING CLAUDIN 18.2

(71) Applicant: LANOVA MEDICINES LIMITED, Shanghai (CN)

(72) Inventors: Ying Qin ZANG, Shanghai (CN); Runsheng LI, Shanghai (CN); Wentao HUANG, Shanghai (CN); Xia QIN, Shanghai (CN); Liang KONG, Shanghai (CN)

(21) Appl. No.: 18/871,070

(22) PCT Filed: Jun. 2, 2023
(86) PCT No.: PCT/CN2023/098030

§ 371 Date: Dec. 2, 2024

(30) Foreign Application Priority Data
Jun. 3, 2022 (WO) PCT/CN2022/097026

(51) Int. Cl. A61K 47/68; A61K 39/00; A61K 39/395; A61P 35/00

(52) U.S. Cl. CPC A61K 47/68031; A61K 39/3955; A61K 47/6849; A61K 47/6889; A61P 35/00; A61K 2039/505

(57) ABSTRACT

Provided are combination therapies that include an inhibitor of PD-1 or PD-L1 and an antibody-drug conjugate targeting claudin 18.2.

### BACKGROUND

Claudins are a family of proteins that form the important components of the tight cell junctions. They establish a paracellular barrier which controls the flow of molecules between the cells. The proteins have N-terminus and a C-terminus in the cytoplasm. Different claudins are expressed on different tissues, their altered function has linked to formation of cancers of respective tissues. Claudin-1 is expressed in colon cancer, claudin-18 is expressed in gastric cancer, and claudin-10 is expressed in hepatocellular carcinoma.

Claudin-18 has two isoforms, isoform 1 and isoform 2. Isoform 2 (Claudin 18.2 or CLDN18.2) is a highly selective cell lineage marker. Claudin 18.2's expression in normal tissues is strictly confined to differentiated epithelial cells of the gastric mucosa, but it was absent from the gastric stem cell zone. Claudin 18.2 was retained on malignant transformation and was expressed in a significant proportion of primary gastric cancers and its metastases. Frequently ectopic activation of claudin 18.2 was also found in pancreatic, esophageal, ovarian, and lung tumors. These data suggested that CLDN18.2 has highly restricted expression pattern in normal tissues, with frequent ectopic activation in a diversity of human cancers.

### SUMMARY

It is discovered herein that an antibody-drug conjugate based on LM-001 was able to synergistically inhibit tumor growth with an anti-PD-1 antibody. This is believed to be at least attributable to LM-001's excellent ability to induce receptor-mediated antibody internalization. Amino acid residues on the claudin 18.2 protein that are important for the binding to LM-001 include those that are important for stabilizing the conformation of the extracellular loops (e.g., W30, L49, W50, C53, C63 and R80). More important, the residues that are involved in binding to the antibodies are contemplated to include N45, Y46, G48, V54, R55, E56, S58, F60, and E62, which are located between the β3 and β4 strands of the first extracellular loop, and Y169 and G172, which are in β5 of the second extracellular loop. By contrast, it is believed that known anti-claudin 18.2 antibodies only bind to one of the extracellular loops.

In accordance with one embodiment of the present disclosure, provided is a method for treating cancer in a patient in need thereof, comprising administering to the patient an inhibitor of PD-1 or PD-L1 and an antibody-drug conjugate (ADC) comprising a drug moiety covalently attached to an anti-claudin 18.2 antibody comprising a VH CDR1, a VH CDR2, a VH CDR3, a VL CDR1, a VL CDR2, and a VL CDR3 which, respectively, comprise the amino acid sequences of SEQ ID NO: 3-8. In some embodiments, the anti-claudin 18.2 antibody comprises a VH comprising the amino acid sequence of SEQ ID NO:1 and a VL comprising the amino acid of SEQ ID NO:2.

In some embodiments, the anti-claudin 18.2 antibody is attached to 2 to 10 of the drug moiety, preferably 2-6 of the drug moiety.

In some embodiments, the drug moiety is a cytotoxic or cytostatic agent. In some embodiments, the drug moiety is a maytansinoid or an auristatin. In some embodiments, the drug moiety comprises DM1 or DM4. In some embodiments, the drug moiety comprises monomethyl auristatin E (MMAE) or monomethyl auristatin F (MMAF).

In some embodiments, the drug moiety is attached to the antibody or fragment thereof through a linker. In some embodiments, the linker is hydrolyzable under acidic conditions. In some embodiments, the linker comprises valine-citrulline.

In some embodiments, the ADC comprises 2 to 6 MMAE or MMAF conjugated to the anti-claudin 18.2 antibody through valine-citrulline.

In some embodiments, the inhibitor of PD-L1 is selected from the group consisting of atezolizumab, avelumab, durvalumab, KN035, CK-301, AUNP12, CA-170 and BMS-986189. In some embodiments, the inhibitor of PD-1 is selected from the group consisting of pembrolizumab, nivolumab, cemiplimab, dostarlimab, JTX-4014, spartalizumab, camrelizumab, sintilimab, tislelizumab, toripalimab, INCMGA00012, AMP-224, and AMP-514. In some embodiments, the inhibitor is toripalimab.

In some embodiments, the inhibitor and the ADC each is administered once every 1 to 6 weeks. In some embodiments, the ADC is administered at a dose from 0.1 mg/kg to 10 mg/kg. In some embodiments, the ADC is administered at a dose of 0.2 mg/kg, 0.4 mg/kg, 0.8 mg/kg, 1.6 mg/kg, 2.0 mg/kg or 2.4 mg/kg.

In some embodiments, the cancer is selected from the group consisting of bladder cancer, liver cancer, colon cancer, rectal cancer, endometrial cancer, leukemia, lymphoma, pancreatic cancer, small cell lung cancer, non-small cell lung cancer, breast cancer, urethral cancer, head and neck cancer, gastrointestinal cancer, stomach cancer, oesophageal cancer, ovarian cancer, renal cancer, melanoma, prostate cancer and thyroid cancer.

In a particular embodiment, provided is a method for treating cancer in a patient in need thereof, comprising administering to the patient an inhibitor of PD-1 or PD-L1 and a vcMMAE-conjugated anti-claudin 18.2 antibody (ADC), wherein: the antibody comprises a VH comprising the amino acid sequence of SEQ ID NO: 1 and a VL comprising the amino acid of SEQ ID NO:2; each antibody is conjugated to 2-6 units of the vcMMAE; the ADC is administered at a dose from 1.2 mg/kg to 1.8 mg/kg; and the cancer is a gastrointestinal cancer.

In some embodiments, the gastrointestinal cancer is gastric cancer (GC) or gastroesophageal junction cancer (GJC). In some embodiments, the inhibitor of PD-1 or PD-L1 is toripalimab. In some embodiments, the inhibitor of PD-1 or PD-L1 is nivolumab.

### BRIEF DESCRIPTION OF THE DRAWINGS

FIG. 1 shows body weight changes of the tumor-bearing mice in different groups. Data points represent group mean body weights. Error bars represent standard error of the mean (SEM).

FIG. 2 shows relative change of body weight of the tumor-bearing mice in different groups. Data points represent group mean relative change body weights. Error bars represent standard error of the mean (SEM)

FIG. 3 shows tumor growth curve of LLC1/H_CLDN18.2 tumor-bearing mice post administration of testing articles. Data points represent group mean, error bars represent standard error of the mean (SEM).

FIG. 4 shows the responses in three human patients treated with LM-002 (1.6 mg/kg or 2.0 mg/kg) and toripalimab (240 mg).

### DETAILED DESCRIPTION

### Definitions

It is to be noted that the term “a” or “an” entity refers to one or more of that entity; for example, “an antibody,” is understood to represent one or more antibodies. As such, the terms “a” (or “an”), “one or more,” and “at least one” can be used interchangeably herein.

As used herein, an “antibody” or “antigen-binding polypeptide” refers to a polypeptide or a polypeptide complex that specifically recognizes and binds to an antigen. An antibody can be a whole antibody and any antigen binding fragment or a single chain thereof. Thus the term “antibody” includes any protein or peptide containing molecule that comprises at least a portion of an immunoglobulin molecule having biological activity of binding to the antigen. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein.

The terms “antibody fragment” or “antigen-binding fragment”, as used herein, is a portion of an antibody such as F(ab′)₂, F(ab)₂, Fab′, Fab, Fv, scFv and the like. Regardless of structure, an antibody fragment binds with the same antigen that is recognized by the intact antibody. The term “antibody fragment” includes aptamers, spiegelmers, and diabodies. The term “antibody fragment” also includes any synthetic or genetically engineered protein that acts like an antibody by binding to a specific antigen to form a complex.

The term antibody encompasses various broad classes of polypeptides that can be distinguished biochemically. Those skilled in the art will appreciate that heavy chains are classified as gamma, mu, alpha, delta, or epsilon (γ, μ, α, δ, ε) with some subclasses among them (e.g., γ1-γ4). It is the nature of this chain that determines the “class” of the antibody as IgG, IgM, IgA IgG, or IgE, respectively. The immunoglobulin subclasses (isotypes) e.g., IgG₁, IgG₂, IgG₃, IgG₄, IgG₅, etc. are well characterized and are known to confer functional specialization. Modified versions of each of these classes and isotypes are readily discernable to the skilled artisan in view of the instant disclosure and, accordingly, are within the scope of the instant disclosure. All immunoglobulin classes are clearly within the scope of the present disclosure, the following discussion will generally be directed to the IgG class of immunoglobulin molecules. With regard to IgG, a standard immunoglobulin molecule comprises two identical light chain polypeptides of molecular weight approximately 23,000 Daltons, and two identical heavy chain polypeptides of molecular weight 53,000-70,000. The four chains are typically joined by disulfide bonds in a “Y” configuration wherein the light chains bracket the heavy chains starting at the mouth of the “Y” and continuing through the variable region.

Antibodies, antigen-binding polypeptides, variants, or derivatives thereof of the disclosure include, but are not limited to, polyclonal, monoclonal, multispecific, human, humanized, primatized, or chimeric antibodies, single chain antibodies, epitope-binding fragments, e.g., Fab, Fab′ and F(ab′)₂, Fd, Fvs, single-chain Fvs (scFv), single-chain antibodies, disulfide-linked Fvs (sdFv), fragments comprising either a VK or VH domain, fragments produced by a Fab expression library, and anti-idiotypic (anti-Id) antibodies (including, e.g., anti-Id antibodies to LIGHT antibodies disclosed herein). Immunoglobulin or antibody molecules of the disclosure can be of any type (e.g., IgG, IgE, IgM, IgD, IgA, and IgY), class (e.g., IgG1, IgG2, IgG3, IgG4, IgAQ1 and IgA2) or subclass of immunoglobulin molecule.

Light chains are classified as either kappa or lambda (κ, λ). Each heavy chain class may be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain.

Both the light and heavy chains are divided into regions of structural and functional homology. The terms “constant” and “variable” are used functionally. In this regard, it will be appreciated that the variable domains of both the light (VK) and heavy (VH) chain portions determine antigen recognition and specificity. Conversely, the constant domains of the light chain (CK) and the heavy chain (CH1, CH2 or CH3) confer important biological properties such as secretion, transplacental mobility, Fc receptor binding, complement binding, and the like. By convention the numbering of the constant region domains increases as they become more distal from the antigen-binding site or amino-terminus of the antibody. The N-terminal portion is a variable region and at the C-terminal portion is a constant region; the CH3 and CK domains actually comprise the carboxy-terminus of the heavy and light chain, respectively.

As indicated above, the variable region allows the antibody to selectively recognize and specifically bind epitopes on antigens. That is, the VK domain and VH domain, or subset of the complementarity determining regions (CDRs), of an antibody combine to form the variable region that defines a three dimensional antigen-binding site. This quaternary antibody structure forms the antigen-binding site present at the end of each arm of the Y. More specifically, the antigen-binding site is defined by three CDRs on each of the VH and VK chains (i.e. CDR-H1, CDR-H2, CDR-H3, CDR-L1, CDR-L2 and CDR-L3). In some instances, e.g., certain immunoglobulin molecules derived from camelid species or engineered based on camelid immunoglobulins, a complete immunoglobulin molecule may consist of heavy chains only, with no light chains. See, e.g., Hamers-Casterman et al., Nature 363:446-448 (1993).

In naturally occurring antibodies, the six “complementarity determining regions” or “CDRs” present in each antigen-binding domain are short, non-contiguous sequences of amino acids that are specifically positioned to form the antigen-binding domain as the antibody assumes its three dimensional configuration in an aqueous environment. The remainder of the amino acids in the antigen-binding domains, referred to as “framework” regions, show less inter-molecular variability. The framework regions largely adopt a β-sheet conformation and the CDRs form loops which connect, and in some cases form part of, the β-sheet structure. Thus, framework regions act to form a scaffold that provides for positioning the CDRs in correct orientation by inter-chain, non-covalent interactions. The antigen-binding domain formed by the positioned CDRs defines a surface complementary to the epitope on the immunoreactive antigen. This complementary surface promotes the non-covalent binding of the antibody to its cognate epitope. The amino acids comprising the CDRs and the framework regions, respectively, can be readily identified for any given heavy or light chain variable region by one of ordinary skill in the art, since they have been precisely defined (see “Sequences of Proteins of Immunological Interest,” Kabat, E., et al., U.S. Department of Health and Human Services, (1983); and Chothia and Lesk, J. Mol. Biol., 196:901-917 (1987)).

By “specifically binds” or “has specificity to,” it is generally meant that an antibody binds to an epitope via its antigen-binding domain, and that the binding entails some complementarity between the antigen-binding domain and the epitope. According to this definition, an antibody is said to “specifically bind” to an epitope when it binds to that epitope, via its antigen-binding domain more readily than it would bind to a random, unrelated epitope. The term “specificity” is used herein to qualify the relative affinity by which a certain antibody binds to a certain epitope. For example, antibody “A” may be deemed to have a higher specificity for a given epitope than antibody “B,” or antibody “A” may be said to bind to epitope “C” with a higher specificity than it has for related epitope “D.”

As used herein, the terms “treat” or “treatment” refer to both therapeutic treatment and prophylactic or preventative measures, wherein the object is to prevent or slow down (lessen) an undesired physiological change or disorder, such as the progression of cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sport, or pet animals such as dogs, cats, guinea pigs, rabbits, rats, mice, horses, cattle, cows, and so on.

As used herein, phrases such as “to a patient in need of treatment” or “a subject in need of treatment” includes subjects, such as mammalian subjects, that would benefit from administration of an antibody or composition of the present disclosure used, e.g., for detection, for a diagnostic procedure and/or for treatment.

### Combination Treatments

It is discovered herein that an antibody-drug conjugate based on LM-001 was able to synergistically inhibit tumor growth with an anti-PD-1 antibody. For instance, 18 days following the treatment, the anti-PD-1 antibody (LM-PD1) alone at 5 mg/kg achieved a tumor growth inhibition (TGI) rate of 10.52% (Table 3); the ADC LM-002 alone was able to achieve a TGI rate of 10.30% (2.5 mg/kg) and 24.45% (5 mg/kg), respectively at two different doses.

When these two agents were used in combination, their TGI rates were 48.65% (2.5 mg/kg LM-PD1 and 2.5 mg/kg LM-002) and 42.63% (2.5 mg/kg LM-PD1 and 5 mg/kg LM-002). Both of these TGI rates were greater than the sum of their individual TGI rates, demonstrating synergism. Also important, while the two different doses of LM-002 led to significant antitumor effects (10.30% vs. 24.45%) as a single agent, there was no such difference for the combinations. This result, therefore, demonstrates that the combination with the anti-PD-1 antibody reduced the requirement for LM-002.

Also interestingly, the combination was tested in human patients with gastric cancer (GC) or gastroesophageal junction cancer (GJC). Again, the combination therapies exhibited significant efficacy, leading to at least one patient to complete response and the rest to partial responses. Surprisingly, the combination with the lower dose of LM-002 (1.6 mg/kg) exhibited better efficacy than the one with the higher dose (2.0 mg/kg), when combined with the same dose (240 mg) of toripalimab, an anti-human PD-1 antibody.

Accordingly, one embodiment of the present disclosure provides a method for treating cancer in a patient in need thereof. In some embodiments, the method entails administering to the patient an inhibitor of PD-1 or PD-L1 and an antibody-drug conjugate (ADC) comprising a drug moiety covalently attached to an anti-claudin 18.2 antibody.

Also provided, in one embodiment, is an inhibitor of PD-1 or PD-L1 for use in treating cancer in a patient, wherein the patient is further treated with an ADC comprising a drug moiety covalently attached to an anti-claudin 18.2 antibody. Also provided is an ADC comprising a drug moiety covalently attached to an anti-claudin 18.2 antibody for use in treating cancer in a patient, wherein the patient is further treated with an inhibitor of PD-1 or PD-L1. Yet another embodiment provides an inhibitor of PD-1 or PD-L1 and an ADC comprising a drug moiety covalently attached to an anti-claudin 18.2 antibody for use in treating cancer.

In some embodiments, the anti-claudin 18.2 antibody includes a VH CDR1, a VH CDR2, a VH CDR3, a VL CDR1, a VL CDR2, and a VL CDR3 which, respectively, comprise the amino acid sequences of SEQ ID NO: 3-8. In some embodiments, the anti-claudin 18.2 antibody includes a VH comprising the amino acid sequence of SEQ ID NO:1 and a VL comprising the amino acid of SEQ ID NO:2.

As demonstrated, the anti-claudin 18.2 antibody LM-001 has excellent ability to induce receptor-mediated antibody internalization even when compared to IMAB362 (claudiximab), a clinical candidate. The greatly increased ability to induce receptor-mediated antibody internalization of the presently disclosed antibodies, it is contemplated, can be attributed to how these antibodies bind to the claudin 18.2 protein. Amino acid residues on the claudin 18.2 protein that are important for the binding to LM-001 include those that are important for stabilizing the conformation of the extracellular loops (e.g., W30, L49, W50, C53, C63 and R80). W30, L49 and W50 are part of the W-LW-C-C consensus motif that helps to stabilize the conformation of loop 1. C53 and C63 form an inter-beta-strand disulfide bond. R80 is likely important for maintaining the interaction between parallel claudin 18.2 molecules on the cell surface, or for stabilizing the conformation of loop 1.

Also important for the antibody binding are residues N45, Y46, G48, V54, R55, E56, S58, F60, E62, Y169 and G172. Among them, N45, Y46, G48, V54, R55, E56, S58, F60 and E62 are located within the β3 strand, or through C63 in the β4 strand. This region, consisting of residues 45-63 of SEQ ID NO:9 (NYQGLWRSCVRESSGFTEC), is hereby referred to as the “β3 to β4 loop,” which is part of the first extracellular loop (loop 1) of claudin 18.2. Y169 and G172, by contrast, are part of the β5 strand (residues 169-172 of SEQ ID NO:3; YTFG) of the second extracellular loop (loop 2).

The greatly increased activity by the presently disclosed antibodies to induce receptor-mediated antibody internalization, it is contemplated, is due to their ability to bind to residues in both the β3 to β4 loop and the β5 strand. In this context, it is believed that known anti-claudin 18.2 antibodies only bind to one of the loops.

### Human Claudin 18.2 Sequence

| Name | Sequence (SEQ ID NO: 9) | |
|---|---|---|
| Human | 1 | MAVTACQGLG FVVSLIGIAG IIAATCMDQW STQDLYNNPV TAVENYQGLW |
| Claudin 18.2 | | ------ |
| (NP_001002026) | 51 | RSCVRESSGF TECRGYFTLL GLPAMLQAVR ALMIVGIVLG AIGLLVSIFA |
| | | -------------- |
| | | β3-β4 loop |
| | 101 | LKCIRIGSME DSAKANMTLT SGIMFIVSGL CAIAGVSVFA NMLVTNFWMS |
| | 151 | TANMYTGMGG MVQTVQTRYT FGAALFVGWV AGGLTLIGGV MMCIACRGLA |
| | | -- -- |
| | | B5 |
| | 201 | PEETNYKAVS YHASGHSVAY KPGGFKASTG FGSNTKNKKI YDGGARTEDE |
| | 251 | VQSYPSKHDY V |

In one embodiment, the ADC includes LM-001 covalently attached to a drug moiety. The drug moiety may be, or be modified to include, a group reactive with a conjugation point on the antibody. For example, a drug moiety can be attached by alkylation (e.g., at the epsilon-amino group lysines or the N-terminus of antibodies), reductive amination of oxidized carbohydrate, transesterification between hydroxyl and carboxyl groups, amidation at amino groups or carboxyl groups, and conjugation to thiols.

In some embodiments, the number of drug moieties, p, conjugated per antibody molecule ranges from an average of 1 to 8; 1 to 7, 1 to 6, 1 to 5, 1 to 4, 1 to 3, or 1 to 2. In some embodiments, p ranges from an average of 2 to 8, 2 to 7, 2 to 6, 2 to 5, 2 to 4 or 2 to 3. In other embodiments, p is an average of 1, 2, 3, 4, 5, 6, 7 or 8. In some embodiments, p ranges from an average of about 1 to about 20, about 1 to about 10, about 2 to about 10, about 2 to about 9, about 1 to about 8, about 1 to about 7, about 1 to about 6, about 1 to about 5, about 1 to about 4, about 1 to about 3, or about 1 to about 2. In some embodiments, p ranges from about 2 to about 8, about 2 to about 7, about 2 to about 6, about 2 to about 5, about 2 to about 4 or about 2 to about 3. In some embodiments, p ranges from 1 to 6, 2 to 5, or 3 to 4.

For example, when chemical activation of the protein results in formation of free thiol groups, the protein may be conjugated with a sulfhydryl reactive agent. In one aspect, the agent is one which is substantially specific for free thiol groups. Such agents include, for example, malemide, haloacetamides (e.g., iodo, bromo or chloro), haloesters (e.g., iodo, bromo or chloro), halomethyl ketones (e.g., iodo, bromo or chloro), benzylic halides (e.g., iodide, bromide or chloride), vinyl sulfone and pyridylthio.

The drug can be linked to the antibody or fragment by a linker. Suitable linkers include, for example, cleavable and non-cleavable linkers. A cleavable linker is typically susceptible to cleavage under intracellular conditions. Suitable cleavable linkers include, for example, a peptide linker cleavable by an intracellular protease, such as lysosomal protease or an endosomal protease. In exemplary embodiments, the linker can be a dipeptide linker, such as a valine-citrulline (val-cit or VC), a phenylalanine-lysine (phe-lys) linker, or maleimidocapronic-valine-citruline-p-aminobenzyloxycarbonyl (mc-Val-Cit-PABA) linker. Another linker is Sulfosuccinimidyl-4-[N-maleimidomethyl]cyclohexane-1-carboxylate (smcc). Sulfo-smcc conjugation occurs via a maleimide group which reacts with sulfhydryls (thiols, —SH), while its Sulfo-NHS ester is reactive toward primary amines (as found in Lysine and the protein or peptide N-terminus). Yet another linker is maleimidocaproyl (mc). Other suitable linkers include linkers hydrolyzable at a specific pH or a pH range, such as a hydrazone linker. Additional suitable cleavable linkers include disulfide linkers. The linker may be covalently bound to the antibody to such an extent that the antibody must be degraded intracellularly in order for the drug to be released e.g. the mc linker and the like.

A linker can include a group for linkage to the antibody. For example, linker can include an amino, hydroxyl, carboxyl or sulfhydryl reactive groups (e.g., malemide, haloacetamides (e.g., iodo, bromo or chloro), haloesters (e.g., iodo, bromo or chloro), halomethyl ketones (e.g., iodo, bromo or chloro), benzylic halides (e.g., iodide, bromide or chloride), vinyl sulfone and pyridylthio).

In some embodiments, the drug moiety is a cytotoxic or cytostatic agent, an immunosuppressive agent, a radioisotope, a toxin, or the like. The conjugate can be used for inhibiting the multiplication of a tumor cell or cancer cell, causing apoptosis in a tumor or cancer cell, or for treating cancer in a patient. The conjugate can be used accordingly in a variety of settings for the treatment of animal cancers. The conjugate can be used to deliver a drug to a tumor cell or cancer cell. Without being bound by theory, in some embodiments, the conjugate binds to or associates with a cancer cell expressing claudin 18.2, and the conjugate and/or drug can be taken up inside a tumor cell or cancer cell through receptor-mediated endocytosis.

Once inside the cell, one or more specific peptide sequences within the conjugate (e.g., in a linker) are hydrolytically cleaved by one or more tumor-cell or cancer-cell-associated proteases, resulting in release of the drug. The released drug is then free to migrate within the cell and induce cytotoxic or cytostatic or other activities. In some embodiments, the drug is cleaved from the antibody outside the tumor cell or cancer cell, and the drug subsequently penetrates the cell, or acts at the cell surface.

Examples of drug moieties or payloads are selected from the group consisting of DM1 (maytansine, N2′-deacetyl-N2′-(3-mercapto-1-oxopropyl)- or N2′-deacetyl-N2′-(3-mercapto-1-oxopropyl)-maytansine), mc-MMAD (6-maleimidocaproyl-monomethylauristatin-D or N-methyl-L-valyl-N-[(1S,2R)-2-methoxy-4-[(2S)-2-[(1R,2R)-1-methoxy-2-methyl-3-oxo-3-[(1S)-2-phenyl-1-(2-thiazolyl)ethyl]amino]propyl]-1-pyr rolidinyl]-1-[(1S)-1-methylpropyl]-4-oxobutyl]-N-methyl-(9Cl)-L-valinamide), mc-MMAF (maleimidocaproyl-monomethylauristatin F or N-[6-(2,5-dihydro-2,5-dioxo-1H-pyrrol-1-yl)-1-oxohexyl]-N-methyl-L-valyl-L-valyl-(3R,4S,5S)-3-methoxy-5-methyl-4-(methylamino) heptanoyl-(αR, βR,2S)-β-methoxy-α-methyl-2-pyrrolidinepropanoyl-L-phenylalanine) and mc-Val-Cit-PABA-MMAE (6-maleimidocaproyl-ValcCit-(p-aminobenzyloxycarbonyl)-monomethylauristatin E or N-[[4-[N-[6-(2,5-dihydro-2,5-dioxo-1H-pyrrol-1-yl)-1-oxohexyl]-L-valyl-N5-(aminocarbonyl)-L-ornithyl]amino]phenyl]methoxy]carbonyl]-N-meth yl-L-valyl-N-[(1S,2R)-4-[(2S)-2-[(1R,2R)-3-[(1R,2S)-2-hydroxy-1-methyl-2-phenylethyl]amino]-1-methoxy-2-methyl-3-oxopropyl]-1-pyrrolidinyl]-2-methoxy-1-[(1S)-1-methylpropyl]-4-oxobutyl]-N-methyl-L-valinamide). DM1 is a derivative of the tubulin inhibitor maytansine while MMAD, MMAE, and MMAF are auristatin derivatives. In some embodiments, the drug moiety is selected from the group consisting of mc-MMAF and mc-Val-Cit-PABA-MMAE. In some embodiments, the drug moiety is a maytansinoid or an auristatin.

In some embodiments, the antibody LM001 is conjugated to MMAE through a Val-Cit linker. In some embodiments, the antibody LM001 is conjugated to MMAF through a Val-Cit linker. In some embodiments, each antibody is conjugated to 1-20, 2-20, 2-8, 2-6, 2-5, or 3-4 of Val-Cit-MMAE or Val-Cit-MMAF.

An inhibitor of PD-1 or PD-L1 refers to any therapeutic agent, small molecule or antibody, that is able to inhibit the activity of PD-1 and PDL1 immune checkpoint proteins present on the surface of cells. PD-1 and PD-L1 inhibitors act to inhibit the association of the programmed death-ligand 1 (PD-L1) with its receptor, programmed cell death protein 1 (PD-1). The interaction of these cell surface proteins is involved in the suppression of the immune system and occurs following infection to limit the killing of bystander host cells and prevent autoimmune disease.

In some embodiment, a PD-1 or PD-L1 inhibitor is anti-PD-1 or anti-PD-L1 antibody. Example PD-1 inhibitors include, without limitation, atezolizumab, avelumab, durvalumab, KN035, CK-301, AUNP12, CA-170 and BMS-986189. Example PD-L1 inhibitors include, without limitation, pembrolizumab, nivolumab, cemiplimab, dostarlimab, JTX-4014, spartalizumab, camrelizumab, sintilimab, tislelizumab, toripalimab, INCMGA00012, AMP-224, and AMP-514.

Pembrolizumab (formerly MK-3475 or lambrolizumab, Keytruda) was developed by Merck and first approved by the Food and Drug Administration in 2014 for the treatment of melanoma. It was later approved for metastatic non-small cell lung cancer and head and neck squamous cell carcinoma. In 2017, it became the first immunotherapy drug approved for use based on the genetic mutations of the tumor rather than the site of the tumor.

Nivolumab (Opdivo) was developed by Bristol-Myers Squibb and first approved by the FDA in 2014 for the treatment of melanoma. It was later approved for squamous cell lung cancer, renal cell carcinoma, and Hodgkin's lymphoma.

Cemiplimab (Libtayo) was developed by Regeneron Pharmaceuticals and first approved by the FDA in 2018 for the treatment of cutaneous squamous cell carcinoma (CSCC) or locally advanced CSCC who are not candidates for curative surgery or curative radiation.

Dostarlimab (Jemperli) was developed by GlaxoSmithKline and was first approved for the treatment of mismatch repair deficient (dMMR) recurrent or advanced endometrial cancer by the FDA in April of 2021. On Aug. 17, 2021, the FDA granted accelerated approval for the treatment of mismatch repair deficient (dMMR) recurrent or advanced solid tumors.

JTX-4014 is being developed by Jounce Therapeutics, and as of 2020 entered Phase I trial. Spartalizumab (PDR001) is a PD-1 inhibitor developed by Novartis to treat both solid tumors and lymphomas, which as of 2018 has entered Phase III trials. Camrelizumab (SHR1210) is an anti-PD-1 monoclonal antibody introduced by Jiangsu HengRui Medicine Co., Ltd. that recently received conditional approval in China for the treatment of relapsed or refractory classical Hodgkin lymphoma.

Sintilimab (IBI308) is a human anti-PD-1 antibody developed by Innovent and Eli Lilly for patients with non-small cell lung cancer (NSCLC). Tislelizumab (BGB-A317) is a humanized IgG4 anti-PD-1 monoclonal antibody in pivotal Phase 3 and Phase 2 clinical trials in solid tumors and hematologic cancers.

Toripalimab (JS 001) is a humanized IgG4 monoclonal antibody against PD-1 under clinical investigation. INCMGA00012 (MGA012) is a humanized IgG4 monoclonal antibody developed by Incyte and MacroGenics. AMP-224 is being developed by AstraZeneca/MedImmune and GlaxoSmithKline. AMP-514 (MEDI0680) is being developed by AstraZeneca.

Atezolizumab (Tecentriq) is a fully humanised IgG1 (immunoglobulin 1) antibody developed by Roche Genentech. In 2016, the FDA approved atezolizumab for urothelial carcinoma and non-small cell lung cancer.

Avelumab (Bavencio) is a fully human IgG1 antibody developed by Merck Serono and Pfizer. Avelumab is FDA approved for the treatment of metastatic merkel-cell carcinoma. It failed phase III clinical trials for gastric cancer.

Durvalumab (Imfinzi) is a fully human IgG1 antibody developed by AstraZeneca. Durvalumab is FDA approved for the treatment of urothelial carcinoma and unresectable non-small cell lung cancer after chemoradiation.

A few more PD-L1 inhibitors are in the experimental phase of development. KN035 is a PD-L1 antibody with subcutaneous formulation currently under clinical evaluations in the US, China, and Japan. CK-301 by Checkpoint Therapeutics is a PD-L1 inhibitor developed by Dana Farber, and is currently in Phase 3 trials for NSCLC.

AUNP12 is a 29-mer peptide as the first peptic PD-1/PD-L1 inhibitor developed by Aurigene and Laboratoires Pierre Fabre that is being evaluated in clinical trial, following promising in vitro results.

CA-170, discovered by Aurigene/Curis as the PD-L1 and VISTA antagonist, was indicted as a potent small molecule inhibitor in vitro. The compound is currently under phase I clinical trial over mesothelioma patients.

BMS-986189 is a macrocyclic peptide discovered by Bristol-Myers Squibb of which the pharmacokinetics, safety and tolerability is currently being studied on healthy subjects.

In a particular embodiment, the PD-1 inhibitor is toripalimab. In another embodiment, the PD-1 inhibitor is nivolumab.

Non-limiting examples of cancers include bladder cancer, breast cancer, colorectal cancer, endometrial cancer, esophageal cancer, head and neck cancer, kidney cancer, leukemia, liver cancer, lung cancer, lymphoma, melanoma, pancreatic cancer, prostate cancer, and thyroid cancer. In some embodiments, the cancer is one or more of gastric, pancreatic, esophageal, ovarian, and lung cancers.

Additional diseases or conditions associated with increased cell survival, that may be treated, prevented, diagnosed and/or prognosed with the antibodies or variants, or derivatives thereof of the disclosure include, but are not limited to, progression, and/or metastases of malignancies and related disorders such as leukemia (including acute leukemias (e.g., acute lymphocytic leukemia, acute myelocytic leukemia (including myeloblastic, promyelocytic, myelomonocytic, monocytic, and erythroleukemia)) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, and solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyo sarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma and retinoblastoma.

A specific dosage and treatment regimen for any particular patient will depend upon a variety of factors, including the particular antibodies, variant or derivative thereof used, the patient's age, body weight, general health, sex, and diet, and the time of administration, rate of excretion, drug combination, and the severity of the particular disease being treated. Judgment of such factors by medical caregivers is within the ordinary skill in the art. The amount will also depend on the individual patient to be treated, the route of administration, the type of formulation, the characteristics of the compound used, the severity of the disease, and the desired effect. The amount used can be determined by pharmacological and pharmacokinetic principles well known in the art.

Methods of administration of the antibodies, fragments, or antibody-drug conjugates or include but are not limited to intradermal, intramuscular, intraperitoneal, intravenous, subcutaneous, intranasal, epidural, and oral routes. The antigen-binding polypeptides or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Thus, pharmaceutical compositions containing the antigen-binding polypeptides of the disclosure may be administered orally, rectally, parenterally, intracistemally, intravaginally, intraperitoneally, topically (as by powders, ointments, drops or transdermal patch), bucally, or as an oral or nasal spray.

The term “parenteral” as used herein refers to modes of administration which include intravenous, intramuscular, intraperitoneal, intrasternal, subcutaneous and intra-articular injection and infusion.

In some embodiments, the PD-L1 or PD-1 inhibitor is administered once every 1, every 2 weeks, or every 3, 4, 5, 6, 7, 8, 9 or 10 weeks. In some embodiments, the ADC is administered once every 1, every 2 weeks, or every 3, 4, 5, 6, 7, 8, 9 or 10 weeks.

In some embodiments, the PD-L1 or PD-1 inhibitor and the ADC are administered on the same day. In some embodiments, the PD-L1 or PD-1 inhibitor and the ADC are administered during the same healthcare visit. In some embodiments, the PD-L1 or PD-1 inhibitor and the ADC are administered separately. In some embodiments, the PD-L1 or PD-1 inhibitor and the ADC are administered in a combined formulation.

In some embodiments, the PD-L1 or PD-1 inhibitor (e.g., toripalimab) is administered at a dose that is about 0.1 mg/kg to 10 mg/kg. In some embodiment, each dose is at least 0.1 mg/kg, 0.2 mg/kg, 0.4 mg/kg, 0.5 mg/kg, 0.8 mg/kg, 1 mg/kg, 1.2 mg/kg, 1.5 mg/kg, 1.6 mg/kg, 1.8 mg/kg, 2 mg/kg, 2.4 mg/kg, 2.5 mg/kg, 2.8 mg/kg, 3 mg/kg, 3.5 mg/kg, 4 mg/kg, or 5 mg/kg. In some embodiment, each dose is not higher than 0.2 mg/kg, 0.4 mg/kg, 0.5 mg/kg, 0.8 mg/kg, 1 mg/kg, 1.2 mg/kg, 1.5 mg/kg, 1.6 mg/kg, 1.8 mg/kg, 2 mg/kg, 2.4 mg/kg, 2.5 mg/kg, 2.8 mg/kg, 3 mg/kg, 3.5 mg/kg, 4 mg/kg, 5 mg/kg or 10 mg/kg. In some embodiment, each dose is 0.2 mg/kg, 0.4 mg/kg, 0.8 mg/kg, 1.6 mg/kg, 2.0 mg/kg or 2.4 mg/kg.

In some embodiments, the ADC is administered at a dose that is about 0.1 mg/kg to 10 mg/kg. In some embodiment, each dose is at least 0.1 mg/kg, 0.2 mg/kg, 0.4 mg/kg, 0.5 mg/kg, 0.8 mg/kg, 1 mg/kg, 1.2 mg/kg, 1.5 mg/kg, 1.6 mg/kg, 1.8 mg/kg, 2 mg/kg, 2.4 mg/kg, 2.5 mg/kg, 2.8 mg/kg, 3 mg/kg, 3.5 mg/kg, 4 mg/kg, or 5 mg/kg. In some embodiment, each dose is not higher than 0.2 mg/kg, 0.4 mg/kg, 0.5 mg/kg, 0.8 mg/kg, 1 mg/kg, 1.2 mg/kg, 1.5 mg/kg, 1.6 mg/kg, 1.8 mg/kg, 2 mg/kg, 2.4 mg/kg, 2.5 mg/kg, 2.8 mg/kg, 3 mg/kg, 3.5 mg/kg, 4 mg/kg, 5 mg/kg or 10 mg/kg. In some embodiment, each dose is 0.2 mg/kg, 0.4 mg/kg, 0.8 mg/kg, 1.6 mg/kg, 2.0 mg/kg or 2.4 mg/kg.

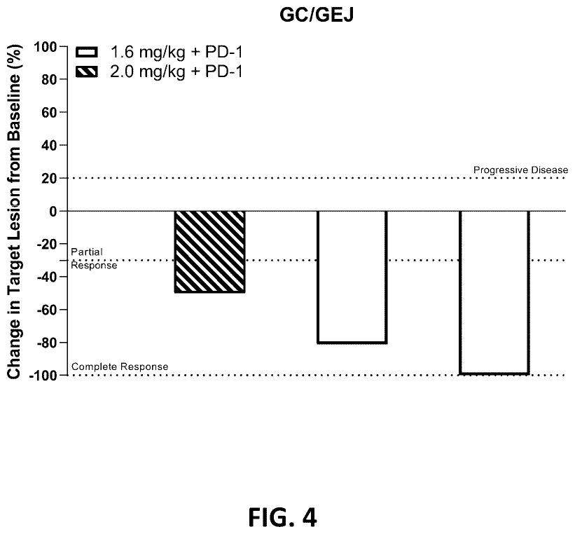

As observed in Example 4 and FIG. 4, higher dose of the ADC does not necessarily lead to higher efficacy. Indeed, among the GC and GEJ patients tested, a lower dose of the ADC (1.6 mg/kg) led to higher (even complete) response than the higher dose (2.0 mg/kg). In some embodiments, therefore, each dose of the ADC is from 1.0 mg/kg to 2.0 mg/kg. In some embodiments, each dose of the ADC is from 1.2 mg/kg to 1.9 mg/kg. In some embodiments, each dose of the ADC is from 1.2 mg/kg to 1.8 mg/kg. In some embodiments, each dose of the ADC is from 1.4 mg/kg to 1.8 mg/kg. In some embodiments, each dose of the ADC is from 1.5 mg/kg to 1.7 mg/kg. In some embodiments, each dose of the ADC is from 1.55 mg/kg to 1.65 mg/kg. In some embodiments, the cancer being treated is a gastrointestinal cancer, such as GC or GEJ.

In a particular embodiment, provided is a method for treating cancer in a patient in need thereof, comprising administering to the patient an inhibitor of PD-1 or PD-L1 and a vcMMAE-conjugated anti-claudin 18.2 antibody (ADC), wherein: the antibody comprises a VH comprising the amino acid sequence of SEQ ID NO:1 and a VL comprising the amino acid of SEQ ID NO:2; each antibody is conjugated to 2-6 units of the vcMMAE; the ADC is administered at a dose from 1.2 mg/kg to 1.8 mg/kg; and the cancer is a gastrointestinal cancer.

In some embodiments, the gastrointestinal cancer is gastric cancer (GC) or gastroesophageal junction cancer (GJC). In some embodiments, the inhibitor of PD-1 or PD-L1 is toripalimab. In some embodiments, the inhibitor of PD-1 or PD-L1 is nivolumab.

### Compositions

The present disclosure also provides pharmaceutical compositions. Such compositions comprise an effective amount of an antibody, fragment, or antibody-drug conjugate, and an acceptable carrier.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. Further, a “pharmaceutically acceptable carrier” will generally be a non-toxic solid, semisolid or liquid filler, diluent, encapsulating material or formulation auxiliary of any type.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose are also envisioned. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences by E. W. Martin, incorporated herein by reference. Such compositions will contain a therapeutically effective amount of the antigen-binding polypeptide, preferably in purified form, together with a suitable amount of carrier so as to provide the form for proper administration to the patient. The formulation should suit the mode of administration. The parental preparation can be enclosed in ampoules, disposable syringes or multiple dose vials made of glass or plastic.

In an embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachet indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

### EXAMPLES

### Example 1: Preparation and Testing of Anti-Claudin 18.2 Antibody Drug Conjugate

This example prepared an antibody drug conjugate (ADC) with antibody 4F11E2 HC N55E-LC S32A (“LM-001”) as disclosed in WO2019219089. LM-001 has the following sequences.

TABLE 1
LM-001 Sequences

| | | SEQ ID |
|---|---|---|
| Name | Sequence | NO: |
| VH | EVQLVESGGGLVQPGGSLRLSCAASGFTFSTFGMHWVRQAPGKGLEWVSYIT | 1 |
| | SGESPIYFTDTVKGRFTISRDNAKNSLYLQMNSLRAEDTAVYYCARSSYYGN | |
| | SMDYWGQGTLVTVSS | |
| VL | DIVMTQSPDSLAVSLGERATINCRSSQSLLNAGNRKNYLTWYQQKPGQPPKL | 2 |
| | LIYWASTRESGVPDRFSGSGSGTDFTLTISSLQAEDVAVYYCQNAYSYPFTF | |
| | GGGTKLEIK | |
| VH CDR1 | TFGMH (246) | 3 |
| VH CDR2 | YITSGESPIYFTDTVKG (311) | 4 |
| VH CDR3 | SSYYGNSMDY (294) | 5 |
| VL CDR1 | RSSQSLLNAGNRKNYLT (309) | 6 |
| VL CDR2 | WASTRES (227) | 7 |
| VL CDR3 | QNAYSYPFT (13) | 8 |

It was determined that residues W30, N45, Y46, G48, L49, W50, C53, V54, R55, E56, S58, F60, E62, C63, R80, Y169, and G172 of the claudin 18.2 protein are involved in the binding to this antibody. W30 is part of a cluster of residues at the first half of the first extracellular domain of the claudin 18.2 protein. N45, Y46, G48, L49, W50, C53, V54, R55, E56, S58, F60, E62 and C63 are in a second cluster of residues within the same extracellular domain. Y169 and G172, on the other hand, are located at or close to the second extracellular domain. The claudin 18.2 protein has four transmembrane segments, a short intracellular N-terminus, a large first extracellular loop (loop 1, or ECS1) that contains a consensus W-LW-C-C motif, a shorter second extracellular loop (loop 2, or ECS2), and an intracellular C-terminal tail. Loop 1 includes four β strands β1, β2, β3, and β4, and loops includes one β strand, β5.

To prepare the ADC (referred to as LM-002), the antibody was mixed with approximately three-fold TCEP and was stirred for 2 h at 37° C. The reaction system was quickly dropped over eight-fold for VC-MMAE and was incubated for 1 h on ice, and 20-fold excess of cysteine was added over the drug linker to extinguish the reaction. Finally, the ADC product was purified by elution through Sephadex G-25 equilibrated in PBS and concentrated by centrifugal ultrafiltration. The conjugate was filtered through a 0.2 μm filter under sterile conditions and stored at −80° C. for analysis and testing. Drug antibody ratio was analyzed by UV spectrometry, the monomer content by SEC-HPLC and free drug content by RP-HPLC. The drug antibody ratio (DAR) of vcMMAE-conjugated LM-001 was 3.76.

### Example 2: Antibody-Drug Conjugate and PD-1 Antibody Combination

This example evaluated the anti-tumor efficacy of LM-002 and anti-PD1 antibody (LM-PD1) individually as single agents and LM-002 in combination with the anti-PD1 antibody, in a C57BL/6 mouse model with LLC1 (Lewis lung carcinoma) cells expressing human CLDN18.2.

Table 2 below shows the experimental design.

TABLE 2
Groups and Treatments for Efficacy Study

| | | Dose level | Dose volume^{b} | Dose | Dose |
|---|---|---|---|---|---|
| Group | N^{a} | (mg/kg) | (mL/kg) | Route | schedule^{c} |
| PBS | 8 | N/A | 10 | i.p. | biw*3 weeks |
| LM-PD1 | 8 | 5 | 10 | i.p. | biw*2 weeks |
| LM-002 | 8 | 2.5 | 5 | i.v. | qw*3 weeks |
| LM-002 | 8 | 5 | 5 | i.v. | qw*3 weeks |
| LM-002 + LM-PD1 | 8 | 2.5 + 5 | 5 + 10 | i.v. + i.p. | qw*3 weeks + biw*2 weeks |
| LM-002 + LM-PD1 | 8 | 5 + 5 | 5 + 10 | i.v. + i.p. | qw*3 weeks + biw*2 weeks |

^{a}Number of animals per group
^{b}Dosing volume: adjust dosing volume based on body weight 5 μL/g or 10 μL/g. Treatment schedule may be adjusted if body weight loss >15%.
^{c}QW: once a week; biw: twice a week

### Cell Culture

The LLC1/H_CLDN18.2 tumor cells were maintained in vitro in DMEM medium supplemented with 10% fetal bovine serum (FBS), 100 U/mL penicillin and 100 μg/mL streptomycin at 37° C. in an atmosphere of 5% CO₂ in air. The tumor cells were routinely sub-cultured twice weekly. The cells growing in an exponential growth phase were harvested and counted for tumor inoculation.

### Tumor Inoculation

Each mouse was inoculated subcutaneously at the right axillary (lateral) with LLC1/H_CLDN18.2 tumor cell (0.3×10⁶) in 0.1 mL of PBS for tumor development. When the tumor volume reached around 50 mm³, which is the 10th day after cell inoculation, the animals were randomly grouped according to tumor volume. LM-002 and LM-PD1 started to dose for the efficacy study. The test article administration and the animal numbers in each group are shown in Table 2.

### Observations

All the procedures related to animal handling, care and the treatment in the study were conducted according to guidelines approved by the Institutional Animal Care and Use Committee (IACUC) of B&K and Medsyin following the guidance of the Association for Assessment and Accreditation of Laboratory Animal Care (AAALAC). At the time of routine monitoring, the animals were daily checked for any effects of tumor growth and treatments on normal behavior such as mobility, food and water consumption (by looking only), body weight gain/loss (body weights were measured every two days or twice weekly), eye/hair matting and any other abnormal effects as stated in the protocol. Death and observed clinical signs were recorded on the basis of the numbers of animals within each subset.

### Tumor Measurements and the Endpoints

The major endpoint is to see if the tumor growth can be delayed or mice can be cured. The experimental endpoint was treated when 1) average tumor volume of control group reached more than 2000 mm³, 2) or mice have been treated 3 weeks. The experiment will be terminated when one condition was satisfied.

Tumor sizes were measured three times weekly in two dimensions using a caliper (No. 105688, sylvac Dantsin), and the volume was expressed in mm³ using the formula: V=0.5a×b² where a and b were the long and short diameters of the tumor, respectively. The tumor sizes were then used for the calculations of T/C (%) values. T/C (%) was calculated using the formula: T/C %=(Ti−T0)/(Vi−V0)×100, Ti was the average tumor volume of a treatment group on a given day, T0 was the average tumor volume of the treatment group on the first day of treatment, Vi was the average tumor volume of the vehicle control group on the same day with Ti, and V0 was the average tumor volume of the vehicle group on the first day of treatment.

TGI (tumor growth inhibition) was calculated for each group using the formula: TGI (%)=[100−T/C]%. According to the guiding principle of anti-tumor drugs of the drug examination center, t/C %≤40%, it is considered that the drug is effective. According to NIH guidelines, TGI %≥58%, the drug is considered to be effective.

### Statistical Analysis

Summary statistics, including mean and the standard error of the mean (SEM), are provided for the tumor volume of each group at each time point (detailed in Section 10.1 Table 6). Statistical analysis of difference in tumor volume among the groups were conducted on the data obtained at the therapeutic time point. P value were calculated by Microsoft Excel 2013.

T-test was performed to compare tumor volume among groups. A value of p<0.05 was considered to be statistically significant.

### Results

The study was completed on day 18 according to the experimental requirement. Details information of tumor volume in different group is showed below.

### Body Weight

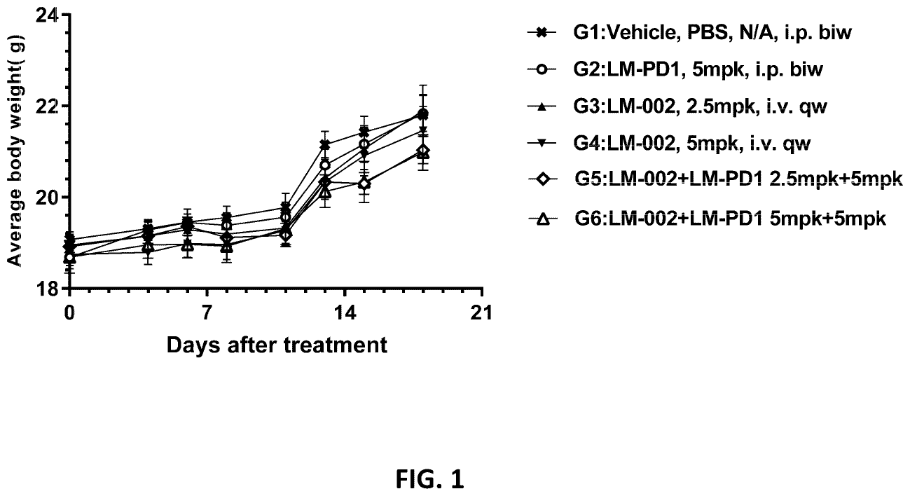

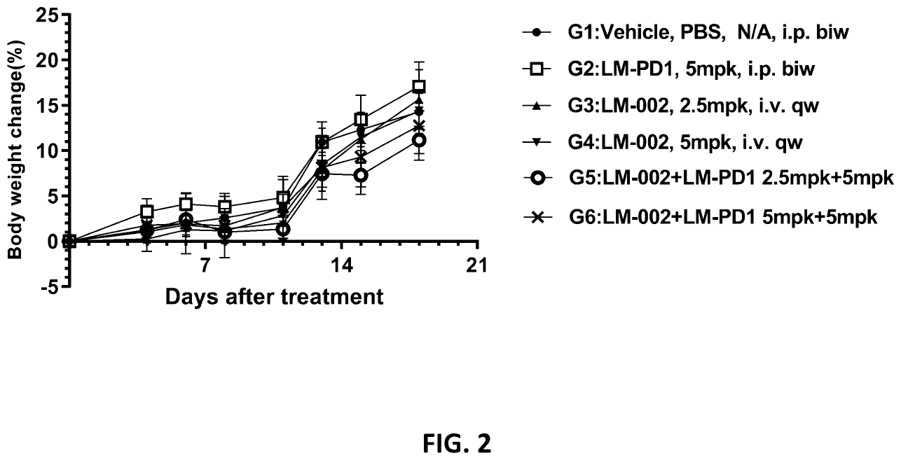

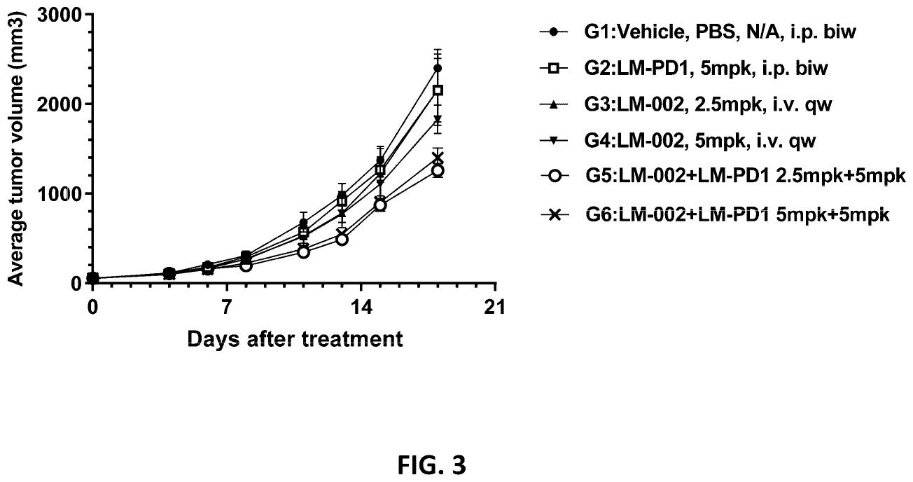

The body weight of LLC1/H_CLDN18.2 tumor bearing mice was monitored regularly as an indirect measure of toxicity. After treatment, the weight of mice in each group did not decrease significantly. The detailed changes of body weight and relative change of body weight of LLC1/H_CLDN18.2 tumor bearing mice after administration are shown in FIG. 1 and FIG. 2. The tumor growth curve of LLC1/H_CLDN18.2 tumor bearing mice after administration is shown in FIG. 3. The data are also shown in Tables 3-4.

TABLE 3
T/C (%), TGI (%) after treatment (N = 8)

| | Days after treatment | | | | | | | |
|---|---|---|---|---|---|---|---|---|
| T/C(%) | 0 | 4 | 6 | 8 | 11 | 13 | 15 | 18 |
| G2: LM-PD1 5 mpk | — | 87.27 | 80.89 | 95.60 | 82.49 | 92.98 | 92.02 | 89.48 |
| G3: LM-002 2.5 mpk | — | 91.50 | 82.97 | 81.98 | 75.78 | 78.41 | 88.81 | 89.70 |
| G4: LM-002 5 mpk | — | 74.72 | 69.45 | 85.66 | 74.92 | 77.54 | 80.05 | 75.55 |
| G5: LM-002 + LM-PD1 | — | 87.46 | 69.48 | 55.75 | 46.43 | 46.78 | 62.01 | 51.35 |
| 2.5 mpk + 5 mpk | | | | | | | | |
| G6: LM-002 + LM-PD1 | — | 75.35 | 65.78 | 68.03 | 52.40 | 53.09 | 64.57 | 57.37 |
| 5 mpk + 5 mpk | | | | | | | | |
| | Days after treatment | | | | | | | |
| TGI(%) | 0 | 4 | 6 | 8 | 11 | 13 | 15 | 18 |
| G2: LM-PD1 5 mpk | — | 12.73 | 19.11 | 4.40 | 17.51 | 7.02 | 7.98 | 10.52 |
| G3: LM-002 2.5 mpk | — | 8.50 | 17.03 | 18.02 | 24.22 | 21.59 | 11.19 | 10.30 |
| G4: LM-002 5 mpk | — | 25.28 | 30.55 | 14.34 | 25.08 | 22.46 | 19.95 | 24.45 |
| G5: LM-002 + LM-PD1 | — | 12.54 | 30.52 | 44.25 | 53.57 | 53.22 | 37.99 | 48.65 |
| 2.5 mpk + 5 mpk | | | | | | | | |
| G6: LM-002 + LM-PD1 | — | 24.65 | 34.22 | 31.97 | 47.60 | 46.91 | 35.43 | 42.63 |
| 5 mpk + 5 mpk | | | | | | | | |

TABLE 4
P value in different group at different time points

| P value vs. | Days after treatment | | | | | | | |
|---|---|---|---|---|---|---|---|---|
| PBS group | 0 | 4 | 6 | 8 | 11 | 13 | 15 | 18 |
| G2: LM-PD1 5 mpk | 0.9977 | 0.6707 | 0.2109 | 0.8730 | 0.4164 | 0.6949 | 0.6253 | 0.5588 |
| G3: LM-002 2.5 mpk | 0.9945 | 0.8139 | 0.4942 | 0.5498 | 0.3525 | 0.3514 | 0.6538 | 0.5988 |
| G4: LM-002 5 mpk | 0.9818 | 0.349 | 0.0844 | 0.6033 | 0.2705 | 0.2125 | 0.2232 | 0.0452 |
| G5: LM-002 + LM-PD1 | 0.9937 | 0.6465 | 0.0675 | 0.0725 | 0.0106 | 0.002 | 0.0116 | 0.0002 |
| 2.5 mpk + 5 mpk | | | | | | | | |
| G6: LM-002 + LM-PD1 | 0.9836 | 0.3783 | 0.0247 | 0.1997 | 0.0302 | 0.007 | 0.0139 | 0.0008 |
| 5 mpk + 5 mpk | | | | | | | | |
| P value | | | | | | | | |
| vs. anti- | Days after treatment | | | | | | | |
| PD1 group | 0 | 4 | 6 | 8 | 11 | 13 | 15 | 18 |
| G5: LM-002 + LM-PD1 | 0.9954 | 0.9930 | 0.4358 | 0.0754 | 0.0085 | 0.0015 | 0.0250 | 0.0277 |
| 2.5 mpk + 5 mpk | | | | | | | | |
| G6: LM-002 + LM-PD1 | 0.9798 | 0.6416 | 0.2364 | 0.2270 | 0.0505 | 0.0073 | 0.0307 | 0.0623 |
| 5 mpk + 5 mpk | | | | | | | | |

This example set out to test the antitumor efficacy of antibodies (LM-002 and LM-PD1) in LLC1/H_CLDN18.2 model. The changes of body weight and relative change of body weight of mice on day 18 after administration are shown in FIG. 1-2. During the administration period, all groups of mice had no significant body weight loss, and the mice in the administration group had good tolerance.

After administration, the average tumor volume of PBS group, LM-PD1 (5 mg/kg) group, LM-002 (2.5 mg/kg) group, LM-002 (5 mg/kg) group, LM-002 (2.5 mg/kg)+LM-PD1 (5 mg/kg) group and LM-002 (5 mg/kg)+LM-PD1 (5 mg/kg) group were 2399.71 mm³, 2153.09 mm³, 2158.26 mm³, 1826.44 mm³, 1259.28 mm³, 1400.26 mm³ respectively (FIG. 3). The T/C of LM-PD1 (5 mg/kg) group, LM-002 (2.5 mg/kg) group, LM-002 (5 mg/kg) group, LM-002 (2.5 mg/kg)+LM-PD1 (5 mg/kg) group and LM-002 (5 mg/kg)+LM-PD1 (5 mg/kg) group were 89.48%, 89.70%, 75.55%, 51.35%, 57.37% respectively. TGI were 10.52%, 10.3%, 25.45%, 48.65%, 42.63% respectively (Table 3). T/C %<40% and TGI %>58% indicate that the treatment is effective.

The p value in treatment groups compared with vehicle group were showed in Table 4. At the end of the experiment (day 18), compared with PBS group, LM-002 at 5 mg/kg and the combination groups showed significant anti-tumor activity, compared with the LM-PD1 single agent group, the combination of LM-002 (2.5 mg/kg)+LM-PD1 (5 mg/kg) showed significantly higher antitumor activity (P=0.0277). Interestingly, the lower dose LM-002 (2.5 mg/kg)+LM-PD1 (5 mg/kg) group had non-inferior anti-tumor effects as compared to the higher dose LM-002 (5 mg/kg)+LM-PD1 (5 mg/kg) group.

### Example 3. Clinical Trial Protocol

The combination of anti-claudin 18.2 antibody-drug conjugate LM-002 and anti-PD-1 antibody toripalimab will be tested in a clinical trial.

Toripalimab will be administrated 240 mg IV, every 3 weeks. LM-002 will be administrated intravenously on day 1 every three weeks. For phase I, there are 2 pre-defined dose levels of LM-002 (0.2 mg/kg, 0.4 mg/kg). For phase Ib, 6 pre-defined dose levels of LM-002 (0.2 mg/kg, 0.4 mg/kg, 0.8 mg/kg, 1.6 mg/kg, 2.0 mg/kg and 2.4 mg/kg) in combination with fixed dose of toripalimab (240 mg).

Patients with advanced solid tumors will be enrolled for this trial.

### Example 4. Clinical Trial Results

The combination of anti-claudin 18.2 antibody-drug conjugate LM-002 (1.6 mg/kg and 2.0 mg/kg) and anti-PD-1 antibody toripalimab (240 mg) was tested in a pilot human trial. Patients being treated had gastric cancer (GC) or gastroesophageal junction cancer (GEJ).

Eight patients were enrolled in this trial, and three patients were evaluable for baseline tumor assessment with at least one post-baseline assessment with target lesions. Their responses are presented in the chart of FIG. 4, which were highly positive. One patient had complete response (CR), and two had partial responses (PR). Interestingly, the combination with the lower dose (1.6 mg/kg) LM-002 was significantly more effective than that with the higher LM-002 dose (2.0 mg/kg).

The present disclosure is not to be limited in scope by the specific embodiments described which are intended as single illustrations of individual aspects of the disclosure, and any compositions or methods which are functionally equivalent are within the scope of this disclosure. It will be apparent to those skilled in the art that various modifications and variations can be made in the methods and compositions of the present disclosure without departing from the spirit or scope of the disclosure. Thus, it is intended that the present disclosure cover the modifications and variations of this disclosure provided they come within the scope of the appended claims and their equivalents.

All publications and patent applications mentioned in this specification are herein incorporated by reference to the same extent as if each individual publication or patent application was specifically and individually indicated to be incorporated by reference.

1. A method for treating cancer in a patient in need thereof, comprising administering to the patient an inhibitor of PD-1 or PD-L1 and an antibody-drug conjugate (ADC) comprising a drug moiety covalently attached to an anti-claudin 18.2 antibody comprising a VH CDR1, a VH CDR2, a VH CDR3, a VL CDR1, a VL CDR2, and a VL CDR3 which, respectively, comprise the amino acid sequences of SEQ ID NO: 3-8.

2. The method of claim 1, wherein the anti-claudin 18.2 antibody comprises a VH comprising the amino acid sequence of SEQ ID NO:1 and a VL comprising the amino acid of SEQ ID NO: 2.

3. The method of claim 1, wherein the anti-claudin 18.2 antibody is attached to 2 to 10 of the drug moiety, preferably 2-6 of the drug moiety, more preferably 3-4 of the drug moiety.

4. The method of claim 1, wherein the drug moiety is a cytotoxic or cytostatic agent.

5. The method of claim 4, wherein the drug moiety is a maytansinoid or an auristatin.

6. The method of claim 5, wherein the drug moiety comprises DM1 or DM4.

7. The method of claim 6, wherein the drug moiety comprises monomethyl auristatin E (MMAE) or monomethyl auristatin F (MMAF).

8. The method of claim 1, wherein the drug moiety is attached to the antibody or fragment thereof through a linker.

9. The method of claim 8, wherein the linker is hydrolyzable under acidic conditions.

10. The method of claim 8, wherein the linker comprises valine-citrulline.

11. The method of claim 1, wherein the ADC comprises 2 to 6 MMAE or MMAF conjugated to the anti-claudin 18.2 antibody through valine-citrulline.

12. The method of claim 1, wherein the inhibitor of PD-L1 is selected from the group consisting of atezolizumab, avelumab, durvalumab, KN035, CK-301, AUNP12, CA-170 and BMS-986189.

13. The method of claim 1, wherein the inhibitor of PD-1 is selected from the group consisting of pembrolizumab, nivolumab, cemiplimab, dostarlimab, JTX-4014, spartalizumab, camrelizumab, sintilimab, tislelizumab, toripalimab, INCMGA00012, AMP-224, and AMP-514.

14. The method of claim 1, wherein the inhibitor is toripalimab.

15. The method of claim 1, wherein the inhibitor and the ADC each is administered once every 1 to 6 weeks.

16. The method of claim 1, wherein the ADC is administered at a dose from 0.1 mg/kg to 10 mg/kg.

17. The method of claim 16, wherein the ADC is administered at a dose of 0.2 mg/kg, 0.4 mg/kg, 0.8 mg/kg, 1.6 mg/kg, 2.0 mg/kg or 2.4 mg/kg.

18. The method of claim 16, wherein the ADC is administered at a dose from 1.2 mg/kg to 1.8 mg/kg.

19. The method of claim 1, wherein the cancer is selected from the group consisting of bladder cancer, liver cancer, colon cancer, rectal cancer, endometrial cancer, leukemia, lymphoma, pancreatic cancer, small cell lung cancer, non-small cell lung cancer, breast cancer, urethral cancer, head and neck cancer, gastrointestinal cancer, stomach cancer, oesophageal cancer, ovarian cancer, renal cancer, melanoma, prostate cancer and thyroid cancer.

20. A method for treating cancer in a patient in need thereof, comprising administering to the patient an inhibitor of PD-1 or PD-L1 and a vcMMAE-conjugated anti-claudin 18.2 antibody (ADC), wherein:
the antibody comprises a VH comprising the amino acid sequence of SEQ ID NO:1 and a VL comprising the amino acid of SEQ ID NO:2;
each antibody is conjugated to 2-6 units of the vcMMAE;
the ADC is administered at a dose from 1.2 mg/kg to 1.8 mg/kg; and
the cancer is a gastrointestinal cancer.

21.-23. (canceled)


SEQUENCE LISTING (9 sequences)

SEQ ID NO: 1 - protein, 119 aa, synthetic construct
EVQLVESGGGLVQPGGSLRLSCAASGFTFSTFGMHWVRQAPGKGLEWVSYITSGESPIYFTDTVKGRFTISRDNAKNSLYLQMNSLRAEDTAVYYCARSSYYGNSMDYWGQGTLVTVSS

SEQ ID NO: 2 - protein, 113 aa, synthetic construct
DIVMTQSPDSLAVSLGERATINCRSSQSLLNAGNRKNYLTWYQQKPGQPPKLLIYWASTRESGVPDRFSGSGSGTDFTLTISSLQAEDVAVYYCQNAYSYPFTFGGGTKLEIK

SEQ ID NO: 3 - protein, 5 aa, synthetic construct
TFGMH

SEQ ID NO: 4 - protein, 17 aa, synthetic construct
YITSGESPIYFTDTVKG

SEQ ID NO: 5 - protein, 10 aa, synthetic construct
SSYYGNSMDY

SEQ ID NO: 6 - protein, 17 aa, synthetic construct
RSSQSLLNAGNRKNYLT

SEQ ID NO: 7 - protein, 7 aa, synthetic construct
WASTRES

SEQ ID NO: 8 - protein, 9 aa, synthetic construct
QNAYSYPFT

SEQ ID NO: 9 - protein, 261 aa, synthetic construct
MAVTACQGLGFVVSLIGIAGIIAATCMDQWSTQDLYNNPVTAVFNYQGLWRSCVRESSGFTECRGYFTLLGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYV